(19) United States
(12) Patent Application Publication
(10) Pub. No.: US 2020/0317523 A1
(43) Pub. Date: Oct. 8, 2020

(54) PRODUCTION OF BETA-TRI-CALCIUM PHOSPHATE (BETA-TCP) WITH HIGH PURITY

(71) Applicant: Yildiz Teknik Universitesi, Istanbul (TR)

(72) Inventors: Afife Binnaz HAZARYORUC, Istanbul (TR); Abdullah TORAMAN, Besiktas/Istanbul (TR)

(73) Assignee: Yildiz Teknik Universitesi, Istanbul (TR)

(21) Appl. No.: 16/957,566

(22) PCT Filed: Dec. 26, 2018
(86) PCT No.: PCT/TR2018/050908

§ 371 Date: Jun. 24, 2020

(30) Foreign Application Priority Data
Dec. 27, 2017 (TR) 2017/22287
Dec. 3, 2018 (TR) 2018/18446

(51) Int. Cl. C01B 25/32; A61L 27/12; A61L 27/58

(52) U.S. Cl. CPC C01B 25/324; A61L 27/12; C01P 2006/80; A61L 2430/02; A61L 27/58

(57) ABSTRACT

The present invention relates to the method of production of beta-tri-calcium phosphate (β-TCP) with high purity and which has an osteoconductive support matrix which can be resorbed and which is bio-compliant when implanted to the defect area and which provides new bone formation in the defect area and which is resorbed while displacing with the newly formed bone in time.

### TECHNICAL FIELD

The present invention relates to the method of production of beta-tri-calcium phosphate (β-TOP) with purity and which has an osteoconductive support matrix which can be resorbed and which is bio-compliant when implanted to the defect area and which provides new bone formation in the defect area and which is resorbed while displacing with the newly formed bone in time.

### KNOWN STATE OF THE ART

Bone and tooth tissues in human body can be damaged due to accident, disease and aging. Therefore, the damaged bones may have to be renewed or changed. Inorganic structure of bone is hydroxyapatite and its basic elements are calcium and phosphate. Hydroxyapatite is joined with collagen and a very strength and flexible structural material is obtained and forms an intravenous dense, metabolically active and viable bone tissue.

Today, hydroxyapatite is used in a stand-alone manner or is used as bone filling substance in regions which do not bear load together with auto-graft. However, in orthopedics and traumatology, in cases where artificial bone tissue is desired to be rapidly dissolved, bio-ceramic materials, which are similar to the composition and structure of the bone tissue and known as β-TCP (beta-tri-calcium phosphate), are used in a stand-alone manner, and bio-ceramic powder, produced in a similar characteristic to the composition of the bone tissue, is the suitable solution for the repair of the defects which occur in the bone tissue.

The minimum criterion in preventing the complications which occur after surgical process and in forming healthy bone tissue is that the purity of β-TCP (Beta-tri-calcium phosphate) powder is greater than 95% and the heavy metal proportions are such that As: ≤0.5 mg/kg, Hg: ≤0.5 mg/kg, Pb: mg/kg. In the present art, there are products which fulfill the defined criteria; however, even these products may lead to important complications after the surgical process. For increasing healthy formation proportion of the bone tissue, purity of β-TCP is increased and the heavy metal proportion is reduced to levels which are lower than the abovementioned values. Since the products with these characteristics do not exist in the medical market, alternative surgical applications cannot be realized.

Another usage area of tri-calcium phosphate is the food sector. Tri-calcium phosphate, which can be used for enriching food products with low cost, also functions as absorbent and as agent which prevents agglomeration and as buffering agent. Tri-calcium phosphate is moreover used for increasing fluidity in foods and as baking agent.

In the known state of the art, there are applications related to synthesizing of the β-TCP (beta-tri-calcium phosphate) powder. One of these is the US patent with number US20030235622A1. In the application, synthesizing of alpha- and beta-tri-calcium phosphate (TCP) powders by using wet chemical method in order to be used as raw substance in artificial bones, artificial joints, and artificial tooth roots and calcium phosphate-based bio-ceramics is disclosed. In said invention, calcium nitrate tetra-hydrate and di-ammonium hydrogen phosphate are used. However, said application relates to a method which is suitable for use in tri-calcium phosphate production having only sub-micron particle size and however, there is no disclosure related to that the tri-calcium phosphate obtained by means of the method disclosed in this application has higher purity. Thus, in the known state of the art, there is no description which will guide the skilled person to obtain high purity tri-calcium phosphate.

As a result, because of all of the abovementioned problems, an improvement is required in the related technical field.

### OBJECT OF THE INVENTION

The present invention relates to high purity beta-tri-calcium phosphate (Beta-TCP) production, for eliminating the above mentioned disadvantages and for bringing new advantages to the related technical field.

The object of the present invention is to realize β-TCP production by using calcium deficient hydroxyapatite (CDHA) synthesized by means of the chemical precipitation method.

The object of the present invention is to realize production of β-TCP powder with high purity (over 99%) from calcium deficient hydroxyapatite (CDHA) by using optimized reaction steps and sintering method.

The object of the present invention is to increase β-TCP (beta-tri-calcium phosphate) powder purity over 95% which is the criterion value determined in formation of healthy bone tissue and in prevention of the complications which will occur after surgical process.

The object of the present invention is to obtain β-TCP (beta-tri-calcium phosphate) having purity under the limit values determined as As: ≤1.5 mg/kg, Hg: ≤1.5 mg/kg, Pb: ≤5 mg/kg in β-TCP (beta-tri-calcium phosphate) powder in the known state of the art.

An object of the present invention is to obtain osteoconductive support matrix which can be resorbed and which is bio-compliant when implanted to the defect area.

An object of the present invention is to provide formation of new bone in the defect area by means of the usage of the subject matter β-TCP (beta-tri-calcium phosphate).

Another object of the present invention is to obtain β-TCP which can be resorbed and which displaces with the newly formed bone in time.

In order to realize all of the abovementioned objects, the present invention is the method of production of beta-tri-calcium phosphate (β-TCP) which has an osteoconductive support matrix which can be resorbed and which is bio-compliant when implanted to the defect area and which provides new bone formation in the defect area and which is resorbed while displacing with the newly formed bone in time, characterized by comprising the steps of:
a. Preparing Ca(OH)₂ solution by adding distilled water onto Ca(OH)₂,
b. Bringing reaction temperature to 25-35° C. and preferably, to 30° C. and continuing mixing,
c. Adjusting reaction pH to the range between 7-9 by using HCl acid,
d. Adding distilled water onto [(NH₄)₂HPO₄] and providing completely dissolving and preparing [(NH₄)₂HPO₄] solution,
e. Adding [(NH₄)₂HPO₄] solution to Ca(OH)₂ solution,
f. Bringing reaction pH to between 7-9 by means of NH₄OH solution and mixing and ending the reaction afterwards,
g. Leaving the obtained solution to aging for between 8 and 30 hours,
h. Adding distilled water onto [(NH₄)₂SO₄] and preparing 3% [(NH₄)₂SO₄] solution,
i. Decanting the aged solution and adding [(NH₄)₂SO₄] solution and leaving for aging for 8-30 hours without mixing,
j. Filtering the obtained solution and washing with plenty of water such that the pH of the solution becomes between approximately 6 and 8, preferably 7,
k. Drying the obtained humid powder in the stove,
l. Sintering the dried powder for 1 hour at a temperature between 900 and 1500° C.,
m. Obtaining the final product by instantaneous cooling of the product at the end of the sintering process.

In an application of the present invention, in step e), [(NH₄)₂HPO₄] solution is added to Ca(OH)₂ solution with flow rate of 3-5 ml/min, for instance, 3 ml/min or 4 ml/min or 5 ml/min.

In an application of the present invention, in step f), the solution whose pH value is adjusted with NH₄OH is mixed for preferably 15 minutes, 20 minutes, 25 minutes, 30 minutes.

In an application of the present invention, in steps g) and i), the solution is preferably left for resting preferably for duration of 12-28 hours, particularly preferably for 15-24 hours.

In an application of the present invention, in step i), preferably 2-4% and particularly preferably 2% or 3% or 4% [(NH₄)₂SO₄] solution is used.

In an application of the present invention, the powder obtained in step k) is dried in stove at a temperature between 70 and 100° C., preferably at 70° C. or 80° C. or 90° C. or 100° C.

The beta-tri-calcium phosphate (β-TCP) obtained by means of said method has at least 99% purity in a different manner from the beta-calcium tri-phosphates (β-TCP) obtained by means of the methods already present in the related art. Moreover, the impurities existing in said beta-calcium tri-phosphate differentiate from the known art by having impurity (Pb ≤1.077, As ≤1.016, Hg ≤1.015) which is substantially lower than the impurity criterion (As: ≤1.5 mg/kg, Hg: ≤1.5 mg/kg, Pb: mg/kg) which is already present in the related art.

From another perspective, the present invention relates to beta-tri-calcium phosphate which has at least 99% purity and which meets the Pb ≤1.077, As ≤1.016, Hg ≤1.015 condition as the impurity limit.

Thus, from one perspective, the present invention relates to beta-calcium tri-phosphate (β-TCP) produced by means of the subject matter method whose details are given above.

In another application of the present invention, the beta-calcium tri-phosphate (β-TCP) obtained by means of the subject matter method is used in a method to be used in the repair of the defects which occur in the bone tissue.

The structural and characteristic properties and all advantages of the present invention will be understood in a clearer manner thanks to the detailed description given below and therefore, evaluation shall be made by taking into consideration this detailed description.

### DETAILED DESCRIPTION OF THE INVENTION

In this detailed description, the method of production of beta-tri-calcium phosphate (β-TCP) with high purity, which has an osteoconductive support matrix which can be resorbed and which is bio-compliant when implanted to the defect area and which provides new bone formation in the defect area and which is resorbed while displacing with the newly formed bone in time, is explained with references to examples without forming any restrictive effect only in order to make the subject more understandable.

In the production of the subject matter high purity beta-tri-calcium phosphate (β-TCP), calcium hydroxide (calcium source) and di-ammonium hydrogen phosphate (phosphate source) are used as the starting substance, and the usage amounts of the starting substances are determined in a stoichiometric manner such that Ca/P proportion is 1.5. The reaction pH is fixed to 7-9 by means of the ammoniac solution and hydro-chloric acid.

Method of production of beta-tri-calcium phosphate (β-TCP) with high purity:
100 ml distilled water is added onto 0.08 mol Ca(OH)₂ and the Ca(OH)₂ solution is prepared,
Reaction temperature is brought to 30° C. and mixing is continued,
Reaction pH is adjusted to 7-9 by using HCl acid,
100 ml distilled water is added onto 0.053 mole [(NH₄)₂HPO₄] and by providing complete dissolving, [(NH₄)₂HPO₄] solution is prepared,
[(NH₄)₂HPO₄] solution is added to the Ca(OH)₂ solution such that 3-5 ml is added in a minute (3-5 ml/minute),
The reaction pH is again brought to between 7-9 by means of NH₄OH solution and is mixed for 20 minutes and the reaction is ended at the end of the duration,
The obtained solution is left for aging for 16 hours,
200 ml distilled water is added onto 6 grams of [(NH₄)₂SO₄] and 3% [(NH₄)₂SO₄] solution is prepared,
The aged solution is decanted and 3% [(NH₄)₂SO₄] solution is added thereon and is again left for aging for 24 hours without mixing,
The obtained solution is filtered by using gooch crucible por: 4 and is washed with plenty of water such that pH is approximately 7,
The obtained humid powder is dried in stove for 24 hours at 90° C.,
The dried powder is sintered for 1 hour at 1100° C.,
The product is instantaneously cooled at the end of the sintering process.

The beta-tri-calcium phosphate obtained by means of the subject matter method has white powder form and its purity is crystal particle size is 73.83 nanometers, size distribution is 1-1000 micrometers, density is 2.973 gr/cm3.

The impurity proportion of beta-tri-calcium phosphate (heavy metals) is such that Pb ≤0.077, As ≤0.016, Zn ≤0.076, Hg ≤0.015, Cr ≤0.085, Co ≤0.07, Ni ≤0.02, Cd ≤0.004, Cu ≤0.13.

The most important point in tissue engineering is to form response for healing of the bone in the defect region. Another important point is that the bone is structurally integrated with the peripheral bone tissue as a result of re-shaping of the bone. Within this scope, the beta-tri-calcium phosphate powder, synthesized by means of the subject matter method, is chemically held onto the surface of the bone tissue (osteointegration) such that no fibrosis tissue is formed in between, supports new bone formation (osteoconduction), stimulates transformation to osteoblastic phenotype in the peripheral tissue (osteoinduction) and forms the new bone cells in the transferred area of the powder (osteogenesis). Moreover, in order for the beta-tri-calcium phosphate powder synthesized by means of the subject matter method to stimulate the new bone cell, the impurity proportion and the chemical composition are substantially important.

Beta-tri-calcium phosphate, synthesized by means of the subject matter method, can also be used as absorbent in the food sector, as baking agent, as agent increasing fluidity and as agent preventing agglomeration in foods.

1. A method to be used in beta-tri-calcium phosphate production, said method is characterized by comprising the steps of:
a. Preparing a Ca(OH)₂ solution comprising distilled water and Ca(OH)₂,
b. Mixing the Ca(OH)₂ solution at a reaction temperature in a range from 25 to 35° C.
c. Adjusting the pH of the Ca(OH)₂ solution to the range between 7-9 by using HCl acid,
d. Preparing a [(NH₄)₂HPO₄] solution comprising distilled water and [(NH₄)₂HPO₄],
e. Adding the [(NH₄)₂HPO₄] solution to the Ca(OH)₂ solution,
f. Bringing the pH of the combined solution to between 7-9 by using aqueous NH₄OH,
g. Leaving the obtained solution to aging for between 8 and 30 hours,
h. Preparing a [(NH₄)₂SO₄] solution comprising distilled water and [(NH₄)₂SO₄],
i. Decanting the aged solution and adding the [(NH₄)₂SO₄] solution and leaving for aging for 8-30 hours without mixing,
j. Filtering the obtained solution and washing with plenty of water such that the pH of the solution becomes between approximately 6 and 8, preferably 7,
k. Drying the obtained humid powder in the stove,
l. Sintering the dried powder for 1 hour at a temperature between 900 and 1500° C.,
m. Obtaining the final product by instantaneous cooling of the product at the end of the sintering process.

2. The method according to claim 1, wherein in step e), the [(NH₄)₂HPO₄] solution is added to the Ca(OH)₂ solution with a flow rate of 3-5 ml/min.

3. The method according to claim 1, wherein step f further comprises mixing the combined solution for 15-30 minutes.

4. The method according to claim 1, wherein in steps g) and i), the solution is left for resting for a duration of 12-28 hours.

5. The method according to claim 1, wherein in step i), the [(NH₄)₂SO₄] solution is a 2-4% [(NH₄)₂SO₄] solution.

6. The method according to claim 1, wherein in step k), the powder is dried at a temperature between 70 and 100° C.

7. (canceled)

8. (canceled)

9. The method according to claim 4, wherein the solution is aged for 15-24 hours.

10. The method according to claim 5, wherein in step i), the [(NH₄)₂SO₄] solution is a 3% [(NH₄)₂SO₄] solution.
